(19) United States
(12) Patent Application Publication
(10) Pub. No.: US 2026/0076635 A1
(43) Pub. Date: Mar. 19, 2026

(54) RADIATION IMAGE PROCESSING DEVICE, RADIATION IMAGE PROCESSING METHOD, AND RADIATION IMAGE PROCESSING PROGRAM

(71) Applicant: FUJIFILM Corporation, Tokyo (JP)

(72) Inventor: Shin HAMAUZU, Kanagawa (JP)

(73) Assignee: FUJIFILM Corporation, Tokyo (JP)

(21) Appl. No.: 19/316,767

(22) Filed: Sep. 2, 2025

(30) Foreign Application Priority Data
Sep. 13, 2024 (JP) 2024-159203

(51) Int. Cl. A61B 6/50; A61B 6/00; A61B 6/46; G06T 7/33

(52) U.S. Cl. CPC A61B 6/505; A61B 6/463; A61B 6/466; A61B 6/5229; A61B 6/5282; G06T 7/337; G06T 2200/04; G06T 2207/10121; G06T 2207/20092; G06T 2207/20224; G06T 2207/30012

(57) ABSTRACT

A processor acquires at least one two-dimensional image including a spine of a subject during surgery on the subject, derives a spine image in which the spine of the subject is emphasized based on the at least one two-dimensional image, extracts a two-dimensional target vertebra that is a target of registration from the two-dimensional image, and registers the two-dimensional target vertebra with a three-dimensional target vertebra that is a target of the registration, the three-dimensional target vertebra being extracted from a three-dimensional image including the spine of the subject acquired before the surgery on the subject.

### CROSS REFERENCE TO RELATED APPLICATIONS

The present application claims priority from Japanese Patent Application No. 2024-159203, filed on Sep. 13, 2024, the entire disclosure of which is incorporated herein by reference.

### BACKGROUND

### Technical Field

The present disclosure relates to a radiation image processing device, a radiation image processing method, and a radiation image processing program.

### Related Art

In spinal surgery, a subject is imaged during surgery using a radiation fluoroscopy device, and a positional relationship between a surgical instrument and a vertebra that is a target of surgery is ascertained using a radiation fluoroscopic image displayed on a display by the imaging. However, while the surgical instrument and the human body structure have a three-dimensional positional relationship, the radiation fluoroscopic image is a two-dimensional image. Even in a case of viewing a two-dimensional radiation fluoroscopic image, it is difficult to ascertain a three-dimensional positional relationship between the surgical instrument and the human body structure.

Therefore, a method has been proposed for registering a three-dimensional image acquired in advance by a computed tomography (CT) apparatus, a magnetic resonance imaging (MRI) apparatus, or the like with a two-dimensional radiation fluoroscopic image (see, for example, JP2019-069037A).

Three-dimensional images are acquired by imaging a subject in a supine position, but spinal surgery is performed with the subject in a prone position. Therefore, the manner in which the spine curves differs between the three-dimensional image and the radiation fluoroscopic image acquired during surgery. Furthermore, because the three-dimensional images are acquired before surgery, the state of gas within the subject and the state of deformation of soft tissues such as internal organs differ between the three-dimensional images and radiation fluoroscopic images acquired during surgery. Therefore, with the method of JP2019-069037A, it is difficult to register a three-dimensional image and a two-dimensional image with high accuracy.

### SUMMARY OF THE INVENTION

The present disclosure has been made in consideration of the above circumstances, and an object of the present disclosure is to perform accurate registration of target vertebrae included in a three-dimensional image and a radiation fluoroscopic image acquired in advance.

According to an aspect of the present disclosure, there is provided a radiation image processing device comprising a processor, in which the processor is configured to: acquire at least one two-dimensional image including a spine of a subject during surgery on the subject; derive a spine image in which the spine of the subject is emphasized based on the at least one two-dimensional image; extract a two-dimensional target vertebra that is a target of registration from the two-dimensional image; and register the two-dimensional target vertebra with a three-dimensional target vertebra that is a target of the registration, the three-dimensional target vertebra being extracted from a three-dimensional image including the spine of the subject acquired before the surgery on the subject.

In the radiation image processing device according to the aspect of the present disclosure, the processor may be configured to: project the three-dimensional target vertebra into two dimensions while changing position and rotation parameters to derive a plurality of projection target vertebra images; and perform the registration by specifying the projection target vertebra images that match the two-dimensional target vertebra.

In the radiation image processing device according to the aspect of the present disclosure, the processor may be configured to: acquire a first two-dimensional image and a second two-dimensional image using radiation having different energy distributions; and derive the spine image by performing weighted subtraction on the first two-dimensional image and the second two-dimensional image.

In the radiation image processing device according to the aspect of the present disclosure, the processor may be configured to: remove scattered ray components from the first two-dimensional image and the second two-dimensional image; and derive the spine image by performing weighted subtraction on the first two-dimensional image and the second two-dimensional image from which the scattered ray components have been removed.

In the radiation image processing device according to the aspect of the present disclosure, the processor may be configured to extract the two-dimensional target vertebra from the two-dimensional image based on a designation by an operator.

In the radiation image processing device according to the aspect of the present disclosure, the processor may be configured to extract the three-dimensional target vertebra from the three-dimensional image.

In the radiation image processing device according to the aspect of the present disclosure, the processor may be configured to extract the three-dimensional target vertebra from the three-dimensional image based on a designation by an operator.

In the radiation image processing device according to the aspect of the present disclosure, the processor may be configured to display the two-dimensional image and an image of the registered three-dimensional target vertebra on a display.

According to another aspect of the present disclosure, there is provided a radiation image processing method executed by a computer, the radiation image processing method comprising: acquiring at least one two-dimensional image including a spine of a subject during surgery on the subject; deriving a spine image in which the spine of the subject is emphasized based on the at least one two-dimensional image; extracting a two-dimensional target vertebra that is a target of registration from the two-dimensional image; and registering the two-dimensional target vertebra with a three-dimensional target vertebra that is a target of the registration, the three-dimensional target vertebra being extracted from a three-dimensional image including the spine of the subject acquired before the surgery on the subject.

According to still another aspect of the present disclosure, there is provided a radiation image processing program causing a computer to execute a procedure comprising: acquiring at least one two-dimensional image including a spine of a subject during surgery on the subject; deriving a spine image in which the spine of the subject is emphasized based on the at least one two-dimensional image; extracting a two-dimensional target vertebra that is a target of registration from the two-dimensional image; and registering the two-dimensional target vertebra with a three-dimensional target vertebra that is a target of the registration, the three-dimensional target vertebra being extracted from a three-dimensional image including the spine of the subject acquired before the surgery on the subject.

The technology of the present disclosure may be provided as a program product.

According to the aspects of the present disclosure, it is possible to perform accurate registration of target vertebrae included in a three-dimensional image and a radiation fluoroscopic image acquired in advance.

### BRIEF DESCRIPTION OF THE DRAWINGS

FIG. 1 is a schematic diagram showing a configuration of a fluoroscopy system including a radiation image processing device according to an embodiment of the present disclosure.

FIG. 2 is a diagram showing a hardware configuration of the radiation image processing device according to the present embodiment.

FIG. 3 is a diagram showing a functional configuration of the radiation image processing device according to the present embodiment.

FIG. 4 is a diagram showing a spine image.

FIG. 5 is a diagram showing a tomographic image of a sagittal cross section of a three-dimensional image.

FIG. 6 is a diagram for describing registration.

FIG. 7 is a diagram showing a display screen.

FIG. 8 is a flowchart showing a process performed in the present embodiment.

### DETAILED DESCRIPTION

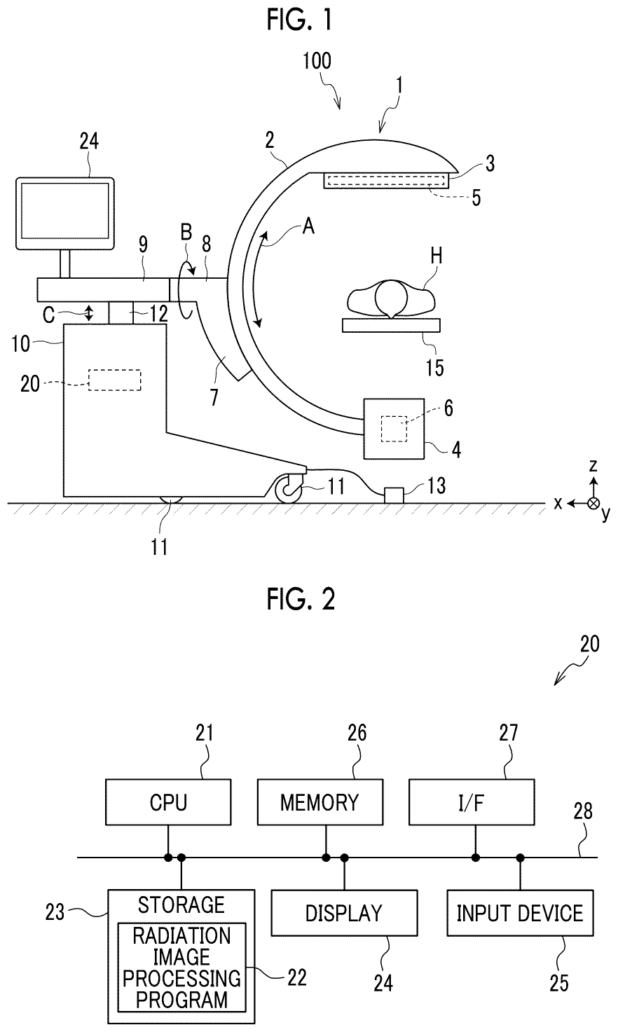

Hereinafter, embodiments of the present disclosure will be described with reference to the drawings. FIG. 1 is a schematic diagram showing a configuration of a fluoroscopy system including a radiation image processing device according to an embodiment of the present disclosure. As shown in FIG. 1, a fluoroscopy system 100 according to the present embodiment comprises a fluoroscopy apparatus 1.

As shown in FIG. 1, the fluoroscopy apparatus 1 according to the present embodiment comprises a C-arm 2. A detection unit 3 is attached to one end part of the C-arm 2, and a radiation emitting unit 4 is attached to the other end part of the C-arm 2 to face the detection unit 3.

The configuration of the fluoroscopy apparatus 1 will be described below in detail. A radiation detector 5, such as a flat panel detector, is provided in the detection unit 3. In addition, for example, a circuit board including a charge amplifier that converts a charge signal read out from the radiation detector 5 into a voltage signal, a sampling two correlation pile circuit that samples the voltage signal output from the charge amplifier, and an analog-digital (AD) conversion unit that converts the voltage signal into a digital signal is also provided in the detection unit 3. Further, in the present embodiment, the radiation detector 5 is used. On the other hand, the present embodiment is not limited to the radiation detector 5 as long as radiation can be detected and the radiation can be converted into an image. For example, a detection device such as an image intensifier can be used.

The radiation detector 5 can repeatedly perform recording and reading out of a radiation image, may be a so-called direct-type radiation detector that directly converts radiation such as X-rays into charges, or may be a so-called indirect-type radiation detector that converts radiation into visible light once and converts the visible light into a charge signal. As a method for reading out a radiation image signal, it is desirable to use the following method: a so-called thin film transistor (TFT) readout method which reads out a radiation image signal by turning on and off a TFT switch; or a so-called optical readout method which reads out a radiation image signal by irradiating a target with readout light. On the other hand, the readout method is not limited thereto, and other methods may be used.

A radiation source 6 is accommodated in the radiation emitting unit 4, and the radiation source 6 emits radiation toward the detection unit 3. The radiation source 6 emits X-rays as radiation, and a timing at which the radiation source 6 emits radiation and a timing at which the radiation detector 5 detects the radiation are controlled by an imaging controller, which will be described later. In addition, the radiation generation conditions in the radiation source 6, that is, the selection of the material of the target and the filter, the tube voltage, the irradiation time, and the like are also controlled by the imaging controller.

The C-arm 2 according to the present embodiment is held by a C-arm holding part 7 to be movable in the direction of an arrow A shown in FIG. 1, and integrally changeable in angle with respect to the detection unit 3 and the radiation emitting unit 4 in a z-axis direction (vertical direction) shown in FIG. 1. In addition, the C-arm holding part 7 includes a shaft part 8, and the shaft part 8 rotatably connects the C-arm 2 to a bearing 9. Thus, the C-arm 2 is configured to be rotatable in the direction of an arrow B shown in FIG. 1 with the shaft part 8 as a rotation axis.

In addition, as shown in FIG. 1, the fluoroscopy apparatus 1 comprises a body part 10. A plurality of wheels 11 are attached to a bottom portion of the body part 10, and thus, the fluoroscopy apparatus 1 can be moved. A support shaft 12 that is expanded and contracted in the z-axis direction of FIG. 1 is provided on an upper portion side of a housing of the body part 10 in FIG. 1. The bearing 9 is held on the upper portion of the support shaft 12 to be movable in the direction of an arrow C. Thus, the C-arm 2 can be moved in an up-down direction with respect to an operating table 15.

In addition, a foot switch 13 for turning on and off the emission of radiation from the radiation source 6 of the radiation emitting unit 4 is connected to the body part 10. A doctor during surgery steps on the foot switch 13 to turn it on, which causes the radiation source 6 to emit radiation in a pulsed manner at a predetermined time interval. In a case in which the doctor removes his/her foot from the foot switch 13, it is turned off, and thus the emission of the radiation from the radiation source 6 is stopped.

The fluoroscopy apparatus 1 has the above-described configuration and performs fluoroscopy of the subject H. That is, the fluoroscopy apparatus 1 irradiates the subject H from below the subject H who is lying prone on the operating table 15 for spinal surgery with radiation, detects the pulsed radiation transmitted through the subject H with the radiation detector 5 of the detection unit 3, and continuously acquires fluoroscopic images of the subject H from the front in accordance with the timing of the emission of the radiation.

Here, the C-arm 2 is movable in the direction of the arrow A, the direction of the arrow B, and the direction of the arrow C, and the fluoroscopy apparatus 1 is movable by the wheels 11. Therefore, the fluoroscopy apparatus 1 can image a desired part of the subject H who is lying prone on the operating table 15 in a desired direction while adjusting their own positions and the position of the C-arm 2.

The body part 10 incorporates the radiation image processing device 20 according to the present embodiment. FIG. 2 is a diagram showing a hardware configuration of the radiation image processing device according to the present embodiment. As shown in FIG. 2, the radiation image processing device 20 is a computer, such as a workstation, a server computer, and a personal computer, and comprises a central processing unit (CPU) 21, a non-volatile storage 23, and a memory 26 as a temporary storage area. In addition, the radiation image processing device 20 comprises a display 24, such as a liquid-crystal display, an input device 25 such as a keyboard and a mouse, and a wired or wireless interface (I/F) 27 that is connected to the detection unit 3, the radiation emitting unit 4, and the foot switch 13, and is used to exchange information with external devices. The CPU 21, the storage 23, the display 24, the input device 25, the memory 26, and the I/F 27 are connected to a bus 28. The CPU 21 is an example of a processor according to the present disclosure.

The storage 23 is realized by a hard disk drive (HDD), a solid-state drive (SSD), a flash memory, and the like. A radiation image processing program 22 installed in the radiation image processing device 20 is stored in the storage 23 serving as a storage medium. The CPU 21 reads out the radiation image processing program 22 from the storage 23, loads the read program into the memory 26, and executes the loaded radiation image processing program 22.

Here, in the present embodiment, a part of a subject H including a surgical target is imaged by a CT apparatus before surgery, thereby acquiring a three-dimensional image V0 including the target part. The three-dimensional image V0 is stored by an external image storage server, but is acquired before surgery by the radiation image processing device 20 according to the present embodiment and is stored in the storage 23. In the present embodiment, it is assumed that the surgical target is the vertebrae that constitute the spine of the subject H.

The radiation image processing program 22 is stored in a storage device of a server computer connected to the network or in a network storage in a state in which it can be accessed from the outside, and is downloaded to and installed on the radiation image processing device 20 in response to a request. Alternatively, the radiation image processing program 22 is recorded on a recording medium, such as a digital versatile disc (DVD) and a compact disc read-only memory (CD-ROM), and distributed, and is installed on the radiation image processing device 20 from the recording medium.

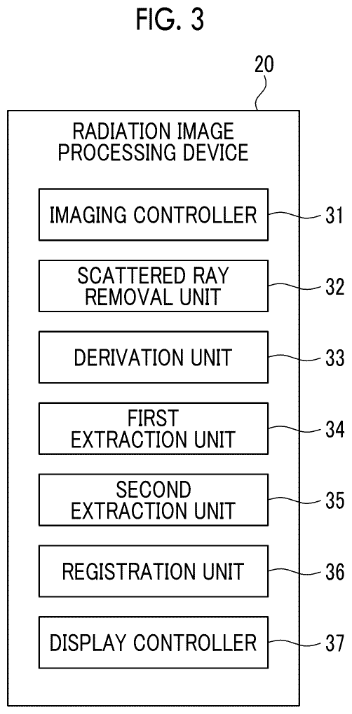

Next, a functional configuration of the radiation image processing device according to the present embodiment will be described. FIG. 3 is a diagram showing the functional configuration of the radiation image processing device according to the present embodiment. As shown in FIG. 3, the radiation image processing device 20 comprises an imaging controller 31, a scattered ray removal unit 32, a derivation unit 33, a first extraction unit 34, a second extraction unit 35, a registration unit 36, and a display controller 37. Then, the CPU 21 executes the radiation image processing program 22, whereby the CPU 21 functions as the imaging controller 31, the scattered ray removal unit 32, the derivation unit 33, the first extraction unit 34, the second extraction unit 35, the registration unit 36, and the display controller 37.

In a case in which the foot switch 13 is turned on and an on signal from the foot switch 13 is input, the imaging controller 31 causes the radiation source 6 included in the radiation emitting unit 4 to emit radiation at a first tube voltage based on preset imaging conditions. Further, the imaging controller 31 detects the radiation transmitted through the subject H with the radiation detector 5 of the detection unit 3 in response to the timing at which the radiation is emitted from the radiation source 6, and generates a fluoroscopic image of the subject H. The generated fluoroscopic image is displayed on the display 24.

In the present embodiment, the imaging controller 31 controls the radiation source 6 to emit the radiation in a pulsed manner at a predetermined interval while the foot switch 13 is turned on. Accordingly, the pulsed radiation is emitted from the radiation source 6, and the fluoroscopic image is generated by the radiation detector 5 at a timing corresponding to the emission of the radiation. Therefore, the fluoroscopic images are continuously displayed on the display 24 like a moving image at a frame rate corresponding to the emission interval of the pulsed radiation.

In a case in which the foot switch 13 is turned off, the imaging controller 31 stores, in the storage 23, a fluoroscopic image (hereinafter referred to as a last image hold (LIH) image) acquired at the last timing at which the foot switch 13 was turned off. Furthermore, in a case in which the foot switch 13 is turned off, the imaging controller 31 causes the radiation source 6 to emit radiation at a second tube voltage different from the first tube voltage, and acquires a second LIH image of the subject H. The LIH image acquired at the first tube voltage is referred to as a first LIH image G1, and the LIH image acquired at the second tube voltage is referred to as a second LIH image G2. The first LIH image G1 and the second LIH image G2 are acquired using different tube voltages, and thus are acquired using radiation with different energy distributions. Note that, although the second tube voltage is higher than the first tube voltage in terms of the first tube voltage and the second tube voltage, this is not intended to be limiting.

Here, each of the first LIH image G1 and the second LIH image G2 includes a scattered ray component based on radiation scattered within the subject H in addition to a primary ray component of radiation that has transmitted through the subject H. Therefore, the scattered ray removal unit 32 removes scattered ray components from the first LIH image G1 and the second LIH image G2. For example, the scattered ray removal unit 32 may remove scattered ray components from the first LIH image G1 and the second LIH image G2 by applying the method disclosed in JP2015-043959A. In a case in which a method disclosed in JP2015-043959A or the like is used, the derivation of the body thickness distribution of the subject H and the scattered ray component for removing the scattered ray component are derived simultaneously.

The removal of scattered ray components from the first LIH image G1 will be described below, but the removal of scattered ray components from the second LIH image G2 can also be performed in a similar manner. First, the scattered ray removal unit 32 acquires a virtual model K of the subject H having an initial body thickness distribution T0(x, y). The virtual model K is data for virtually representing the subject H, in which the body thickness according to the initial body thickness distribution T0(x, y) is associated with the coordinate position of each pixel of the first LIH image G1. Note that the virtual model K of the subject H having the initial body thickness distribution T0(x, y) may be stored in advance in the storage 23. Furthermore, the body thickness distribution T(x, y) of the subject H may be calculated based on a source image receptor distance (SID) that is the distance between the radiation source 6 and the surface of the radiation detector 5 in the fluoroscopy apparatus 1, and a source object distance (SOD) that is the distance between the radiation source 6 and the surface of the subject H. In this case, the body thickness distribution can be obtained by subtracting the SOD from the SID.

Next, the scattered ray removal unit 32 generates an estimated image based on the virtual model K by combining an estimated primary ray image obtained by estimating the primary ray image obtained by imaging the virtual model K and an estimated scattered ray image obtained by estimating the scattered ray image obtained by imaging the virtual model K, as an estimated image estimating the first LIH image G1 obtained by imaging the subject H.

Next, the scattered ray removal unit 32 corrects the initial body thickness distribution T0(x, y) of the virtual model K such that the difference between the estimated image and the first LIH image G1 is reduced. The scattered ray removal unit 32 repeatedly generates the estimated image and corrects the body thickness distribution until the difference between the estimated image and the first LIH image G1 satisfies a predetermined end condition. The scattered ray removal unit 32 derives the body thickness distribution in a case in which the end condition is satisfied as the body thickness distribution T(x, y) of the subject H. Further, the scattered ray removal unit 32 removes the scattered ray components included in the first LIH image G1 by subtracting the scattered ray components in a case in which the end condition is satisfied from the first LIH image G1. In the following description, it is assumed that the first LIH image G1 and the second LIH image G2 have scattered ray components removed.

Here, in the fluoroscopy apparatus 1, since the distance between the subject H and the radiation detector 5 is relatively large, air is interposed between the subject H and the radiation detector 5. Air has unique radiation characteristics. Therefore, by transmitting through air, the radiation quality of the primary ray component and the scattered ray component transmitted through the subject H changes depending on the radiation characteristics of the air. Therefore, in the present embodiment, in a case of removing the scattered ray components, it is preferable to take into consideration the radiation characteristics of the air interposed between the subject H and the radiation detector 5.

As a method for removing scattered ray by taking into account the radiation characteristics of air, for example, the method disclosed in WO2021/100413A can be used. Specifically, the primary ray transmittance and the scattered ray transmittance of radiation for the air interposed between the subject H and the radiation detector 5 are generated in advance as a table or the like according to various imaging conditions and the body thickness distribution of the subject H, and are stored in the storage 23.

In a case in which the scattered ray removal unit 32 estimates the body thickness distribution of the subject H and removes scattered ray, the scattered ray removal unit 32 refers to the table to acquire the radiation characteristics of the air corresponding to the body thickness distribution, that is, the primary ray transmittance and the scattered ray transmittance of the radiation. In addition, the scattered ray removal unit 32 acquires an estimated primary ray image and an estimated scattered ray image using the acquired radiation characteristics, imaging conditions, and body thickness distribution, and generates an estimated image by adding the estimated primary ray image and the estimated scattered ray image. Further, the scattered ray removal unit 32 repeatedly generates the estimated image and corrects the body thickness distribution until the difference between the estimated image and the first LIH image G1 satisfies a predetermined end condition.

Then, the scattered ray removal unit 32 removes the scattered ray components from the first LIH image G1 by subtracting the estimated scattered ray image obtained in a case in which the body thickness distribution that satisfies the end condition is acquired from the first LIH image G1. Accordingly, it is possible to remove scattered ray components from the first LIH image G1 while also taking into consideration the radiation characteristics of the object interposed between the subject H and the radiation detector 5. Similarly, scattered ray components can also be removed from the second LIH image G2.

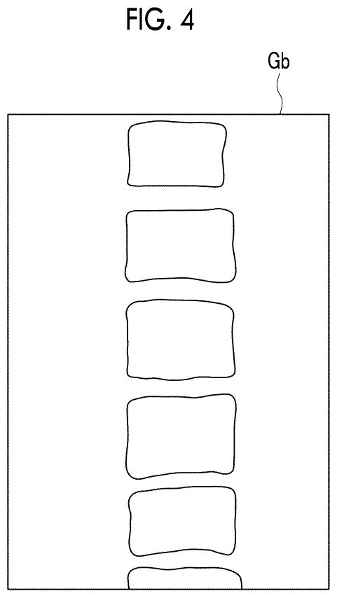

The derivation unit 33 performs an energy subtraction process to derive a spine image Gb in which the spine of the subject H is extracted from the first LIH image G1 and the second LIH image G2. In a case of deriving the spine image Gb, the derivation unit 33 performs weighted subtraction between corresponding pixels of the first LIH image G1 and the second LIH image G2 as shown in the following Equation (1), thereby deriving a spine image Gb in which the spine of the subject H included in each of the LIH image G1 and G2 is extracted, as shown in FIG. 4. In Equation (1), β1 is a weighting coefficient.

$$Gb ( x , y ) = G 1 ( x , y ) - β1 \times G 2 ( x , y ) \qquad ( 1 )$$

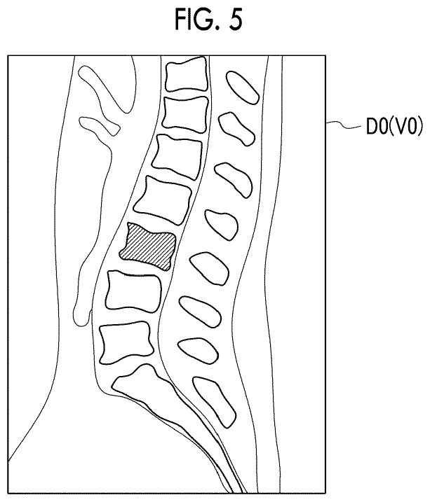

The first extraction unit 34 extracts a target vertebra that is a target of surgery from the three-dimensional image V0. In the present embodiment, the first extraction unit 34 causes the display controller 37 to display, on the display 24, a tomographic image of a sagittal cross section passing through the spine in the three-dimensional image V0. FIG. 5 is a diagram showing a tomographic image of a sagittal cross section displayed on the display 24. The operator uses the input device 25 to designate a target vertebra in a sagittal image D0 displayed on the display 24. In FIG. 5, the designation of the fifth lumbar vertebra is indicated by hatching the fifth lumbar vertebra. The first extraction unit 34 extracts the designated target vertebra from the three-dimensional image V0. The target vertebra extracted from the three-dimensional image V0 by the first extraction unit 34 is hereinafter referred to as a three-dimensional target vertebra T3.

Note that the extraction of the target vertebra by the first extraction unit 34 is not limited to the designation by the operator. For example, the name of the target vertebra may be input by the operator via the input device 25, and the designated vertebra may be extracted as the target vertebra using an extraction model that has been machine-learned to extract a specific vertebra from the three-dimensional image V0.

The second extraction unit 35 extracts a target vertebra that is a target of surgery from the spine image Gb. In the present embodiment, the second extraction unit 35 causes the display controller 37 to display the spine image Gb on the display 24. The operator uses the input device 25 to designate a target vertebra in the spine image Gb displayed on the display 24. The second extraction unit 35 extracts the designated target vertebra from the spine image Gb. The target vertebra extracted from the spine image Gb by the second extraction unit 35 is hereinafter referred to as a two-dimensional target vertebra T2.

Note that the extraction of the target vertebra by the second extraction unit 35 is not limited to the designation by the operator. For example, the name of the target vertebra may be input by the operator via the input device 25, and the designated vertebra may be extracted as the target vertebra using an extraction model that has been machine-learned to extract a specific vertebra from the spine image Gb.

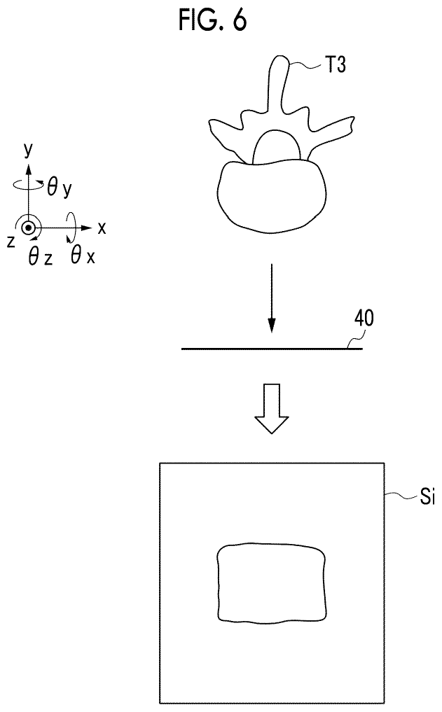

The registration unit 36 registers the three-dimensional target vertebra T3 with the two-dimensional target vertebra T2. Therefore, the registration unit 36 projects the three-dimensional target vertebra T3 into two dimensions while changing the position and rotation parameters, and derives a plurality of projection target vertebra images Si (i=1 to n:n is the number of parameters) having different parameters. FIG. 6 is a diagram for describing the derivation of a projection target vertebra image. The registration unit 36 sets an x-axis in the left-right direction of the human body, a y-axis in the front-rear direction of the human body, and a z-axis in the up-down direction of the human body for the three-dimensional target vertebra T3. Then, the registration unit 36 projects the three-dimensional target vertebra T3 onto a projection surface 40 on the xz plane, as shown by the arrows in FIG. 6, while changing a position parameter tx in an x direction, a position parameter ty in a y direction, a position parameter tz in a z direction, a rotation parameter θx about the x axis, a rotation parameter θy about the y axis, and a rotation parameter θz about the z axis. Accordingly, a projection target vertebra image Si is derived.

The registration unit 36 specifies a projection target vertebra image St that most closely matches the two-dimensional target vertebra T2 from among the plurality of projection target vertebra images Si. For example, the projection target vertebra image St is specified by obtaining a correlation between each of the plurality of projection target vertebra images Si and the two-dimensional target vertebra T2. Accordingly, the registration is completed.

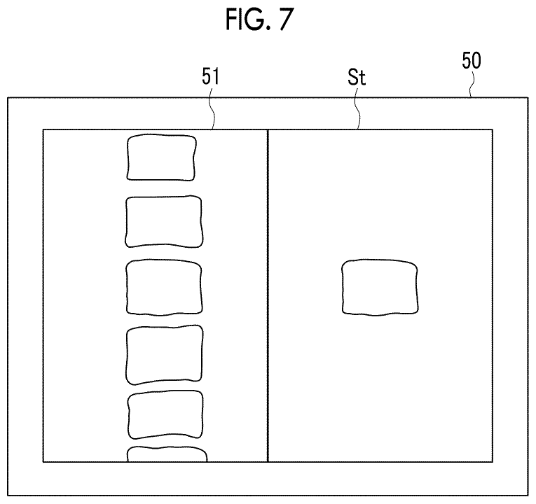

The display controller 37 displays the projection target vertebra image St alongside the fluoroscopic image. FIG. 7 is a diagram showing a display screen according to the present embodiment. As shown in FIG. 7, a fluoroscopic image 51 and a projection target vertebra image St are displayed on a display screen 50. The fluoroscopic image 51 is the first LIH image G1. By comparing the first LIH image G1 with a projection target vertebra image St, the operator, that is, the doctor, can easily ascertain the three-dimensional position and inclination within the subject H of the target vertebra included in the fluoroscopic image 51 displayed as the surgery proceeds.

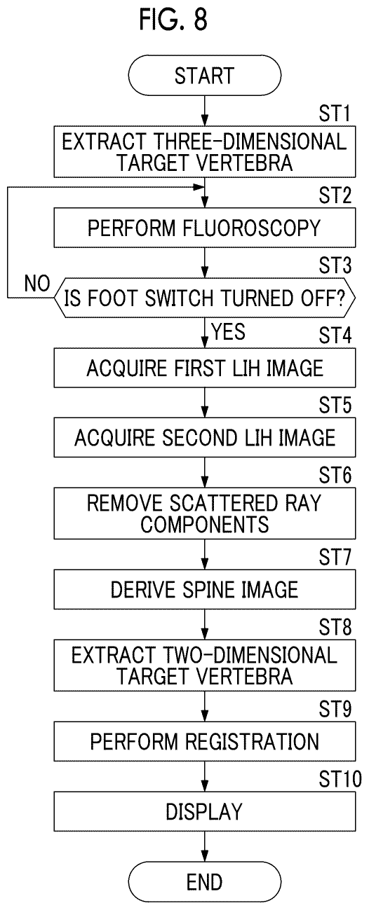

Next, processing performed in the present embodiment will be described. FIG. 8 is a flowchart showing a process performed in the present embodiment. It is assumed that the three-dimensional image V0 is acquired before the surgery and is stored in the storage 23. First, the first extraction unit 34 before the surgery displays the three-dimensional image V0 on the display 24, and extracts a three-dimensional target vertebra T3 according to an instruction from the operator (Step ST1).

Subsequently, in a case in which the foot switch 13 is turned on, the imaging controller 31 performs fluoroscopy of the subject H (Step ST2), and starts monitoring whether or not the foot switch 13 is turned off (Step ST3). In a case in which determination in Step ST3 is positive, the imaging controller 31 acquires the first LIH image G1 (Step ST4), and then acquires the second LIH image G2 (Step ST5).

The scattered ray removal unit 32 removes scattered ray components from the first LIH image G1 and the second LIH image G2 (Step ST6), and the derivation unit 33 derives a spine image Gb based on the first LIH image G1 and the second LIH image G2 from which the scattered ray components have been removed (Step ST7). Subsequently, the second extraction unit 35 displays the spine image Gb on the display 24, and extracts a two-dimensional target vertebra T2 according to an instruction from the operator (Step ST8).

The registration unit 36 then performs registration between the three-dimensional target vertebra T3 and the two-dimensional target vertebra T2, and derives a projection target vertebra image St (Step ST9). Furthermore, the display controller 37 displays the fluoroscopic image (first LIH image G1) and the projection target vertebra image St on the display 24 (Step ST10), and the process ends.

In this manner, in the present embodiment, registration between the three-dimensional target vertebra T3 extracted from the three-dimensional image V0 and the two-dimensional target vertebra T2 is performed. Therefore, even in a case in which the manner in which the spine curves differs between a case where the three-dimensional image V0 is acquired and a case where surgery is performed, or the state of gas and soft tissue within the subject H differs, the target vertebrae included in the three-dimensional image V0 can be registered with the target vertebrae included in the fluoroscopic image with high accuracy. Therefore, by displaying the registered projection target vertebra image St and the fluoroscopic image, the operator can easily check the three-dimensional position and state of the target vertebra in the fluoroscopic image.

In addition, in the present embodiment, the projection target vertebra image Si is derived while changing the position and rotation parameters of the three-dimensional target vertebra T3 extracted from the three-dimensional image V0, and the registration is performed. Therefore, the registration process can be performed faster than in a case in which registration is performed while changing the overall parameters of the three-dimensional image V0.

In the above embodiment, the three-dimensional target vertebra T3 is extracted from the three-dimensional image V0 in the radiation image processing device 20 according to the present embodiment, but the present disclosure is not limited thereto. The three-dimensional target vertebra T3 extracted from the three-dimensional image V0 may be stored in advance in the storage 23, and the stored three-dimensional target vertebra T3 may be used to perform the registration.

In addition, in the above embodiment, the radiation is not particularly limited, and for example, α-rays or γ-rays other than X-rays can be applied.

In the present embodiment, each process is executed by any computer. Further, any computer may execute these processes using a processor as hardware, a program as software, or a combination thereof. In this case, the processor is configured to cooperate with the program to execute various processes in the present embodiment, and can function as each unit or each means in the present embodiment. Further, the order in which the processes are executed by the processor is not limited to the order described above and may be changed as appropriate. Any computer may be a general purpose computer, a special purpose computer, a workstation, or other system capable of executing each process.

The processor may be configured with one or more pieces of hardware, and the type of hardware is not limited. For example, the processor may be configured with hardware such as a central processing unit (CPU), a micro processing unit (MPU), a programmable logic device such as a field-programmable gate array (FPGA), a dedicated circuit for executing specific processing such as an application-specific integrated circuit (ASIC), a graphic processing unit (GPU), or a neural processing unit (NPU). Additionally, the types of hardware may be a combination of different types of hardware. In a case in which a plurality of pieces of hardware are configured to execute one or more processes of a certain processor, the plurality of pieces of hardware may be present in devices physically separate from each other, or may be present in the same device. In addition, in any of the embodiments, the order of the processes performed by the processor is not limited to the order described above, and may be changed as appropriate. The hardware is configured by electrical circuits (circuitry) combining circuit elements such as semiconductor elements.

Further, the program may be software such as firmware or a microcode. The program may also be, for example, a group of program modules, each function of which may be implemented by a processor configured to execute each function. The program may be a program code or a number of code segments stored in one or more non-transitory computer-readable media (for example, storage media or other storages). The program may be stored in a plurality of non-transitory computer-readable media that are present in apparatuses physically separate from each other in a divided manner. A Program code or a code segment may represent a procedure, a function, a subprogram, a routine, a subroutine, a module, a software package, a class, or any combination of instructions, data structures, or program statements. A program code or a code segment may be connected to another code segment or a hardware circuit by transmitting and receiving information, data, arguments, parameters, or memory content.

In the above embodiment, the radiation image processing program 22 is stored (installed) in the storage 23 in advance, but the present disclosure is not limited thereto. The information processing program 30 may be provided in a form recorded in a recording medium such as a compact disc read-only memory (CD-ROM), a digital versatile disc read-only memory (DVD-ROM), and a universal serial bus (USB) memory. In addition, the radiation image processing program 22 may be configured to be downloaded from an external device via a network.

The technology of the present disclosure is applied to any program products. The program product includes products in all aspects for providing a program. For example, the program product includes a program provided through a network such as the Internet, and a non-transitory computer readable recording medium such as a CD-ROM, a DVD, or a USB memory on which the program is stored.

The supplementary notes of the present disclosure will be described below.

### Supplementary Note 1

A radiation image processing device comprising a processor,
in which the processor is configured to:
   acquire at least one two-dimensional image including a spine of a subject during surgery on the subject;
   derive a spine image in which the spine of the subject is emphasized based on the at least one two-dimensional image;
   extract a two-dimensional target vertebra that is a target of registration from the two-dimensional image; and
   register the two-dimensional target vertebra with a three-dimensional target vertebra that is a target of the registration, the three-dimensional target vertebra being extracted from a three-dimensional image including the spine of the subject acquired before the surgery on the subject.

### Supplementary Note 2

The radiation image processing device according to Supplementary Note 1, in which the processor is configured to:
project the three-dimensional target vertebra into two dimensions while changing position and rotation parameters to derive a plurality of projection target vertebra images; and
perform the registration by specifying the projection target vertebra images that match the two-dimensional target vertebra.

### Supplementary Note 3

The radiation image processing device according to Supplementary Note 1 or 2, in which the processor is configured to:
acquire a first two-dimensional image and a second two-dimensional image using radiation having different energy distributions; and
derive the spine image by performing weighted subtraction on the first two-dimensional image and the second two-dimensional image.

### Supplementary Note 4

The radiation image processing device according to Supplementary Note 3, in which the processor is configured to:
remove scattered ray components from the first two-dimensional image and the second two-dimensional image; and
derive the spine image by performing weighted subtraction on the first two-dimensional image and the second two-dimensional image from which the scattered ray components have been removed.

### Supplementary Note 5

The radiation image processing device according to any one of Supplementary Notes 1to 4, in which the processor is configured to extract the two-dimensional target vertebra from the two-dimensional image based on a designation by an operator.

### Supplementary Note 6

The radiation image processing device according to any one of Supplementary Notes 1to 5, in which the processor is configured to extract the three-dimensional target vertebra from the three-dimensional image.

### Supplementary Note 7

The radiation image processing device according to Supplementary Note 6, in which the processor is configured to extract the three-dimensional target vertebra from the three-dimensional image based on a designation by an operator.

### Supplementary Note 8

The radiation image processing device according to any one of Supplementary Notes 1to 7, in which the processor is configured to display the two-dimensional image and an image of the registered three-dimensional target vertebra on a display.

### Supplementary Note 9

A radiation image processing method executed by a computer, the radiation image processing method comprising:
acquiring at least one two-dimensional image including a spine of a subject during surgery on the subject;
deriving a spine image in which the spine of the subject is emphasized based on the at least one two-dimensional image;
extracting a two-dimensional target vertebra that is a target of registration from the two-dimensional image; and
registering the two-dimensional target vertebra with a three-dimensional target vertebra that is a target of the registration, the three-dimensional target vertebra being extracted from a three-dimensional image including the spine of the subject acquired before the surgery on the subject.

### Supplementary Note 10

A radiation image processing program causing a computer to execute a procedure comprising:
acquiring at least one two-dimensional image including a spine of a subject during surgery on the subject;
deriving a spine image in which the spine of the subject is emphasized based on the at least one two-dimensional image;
extracting a two-dimensional target vertebra that is a target of registration from the two-dimensional image; and
registering the two-dimensional target vertebra with a three-dimensional target vertebra that is a target of the registration, the three-dimensional target vertebra being extracted from a three-dimensional image including the spine of the subject acquired before the surgery on the subject.

What is claimed is:

1. A radiation image processing device comprising a processor,
wherein the processor is configured to:
acquire at least one two-dimensional image including a spine of a subject during surgery on the subject;
derive a spine image in which the spine of the subject is emphasized based on the at least one two-dimensional image;
extract a two-dimensional target vertebra that is a target of registration from the two-dimensional image; and
register the two-dimensional target vertebra with a three-dimensional target vertebra that is a target of the registration, the three-dimensional target vertebra being extracted from a three-dimensional image including the spine of the subject acquired before the surgery on the subject.

2. The radiation image processing device according to claim 1, wherein the processor is configured to:
project the three-dimensional target vertebra into two dimensions while changing position and rotation parameters to derive a plurality of projection target vertebra images; and
perform the registration by specifying the projection target vertebra images that match the two-dimensional target vertebra.

3. The radiation image processing device according to claim 1, wherein the processor is configured to:
acquire a first two-dimensional image and a second two-dimensional image using radiation having different energy distributions; and
derive the spine image by performing weighted subtraction on the first two-dimensional image and the second two-dimensional image.

4. The radiation image processing device according to claim 3, wherein the processor is configured to:
remove scattered ray components from the first two-dimensional image and the second two-dimensional image; and
derive the spine image by performing weighted subtraction on the first two-dimensional image and the second two-dimensional image from which the scattered ray components have been removed.

5. The radiation image processing device according to claim 1, wherein the processor is configured to extract the two-dimensional target vertebra from the two-dimensional image based on a designation by an operator.

6. The radiation image processing device according to claim 1, wherein the processor is configured to extract the three-dimensional target vertebra from the three-dimensional image.

7. The radiation image processing device according to claim 6, wherein the processor is configured to extract the three-dimensional target vertebra from the three-dimensional image based on a designation by an operator.

8. The radiation image processing device according to claim 1, wherein the processor is configured to display the two-dimensional image and an image of the registered three-dimensional target vertebra on a display.

9. A radiation image processing method executed by a computer, the radiation image processing method comprising:
acquiring at least one two-dimensional image including a spine of a subject during surgery on the subject;
deriving a spine image in which the spine of the subject is emphasized based on the at least one two-dimensional image;
extracting a two-dimensional target vertebra that is a target of registration from the two-dimensional image; and
registering the two-dimensional target vertebra with a three-dimensional target vertebra that is a target of the registration, the three-dimensional target vertebra being extracted from a three-dimensional image including the spine of the subject acquired before the surgery on the subject.

10. A non-transitory computer-readable storage medium that stores a radiation image processing program causing a computer to execute a procedure comprising:
acquiring at least one two-dimensional image including a spine of a subject during surgery on the subject;
deriving a spine image in which the spine of the subject is emphasized based on the at least one two-dimensional image;
extracting a two-dimensional target vertebra that is a target of registration from the two-dimensional image; and
registering the two-dimensional target vertebra with a three-dimensional target vertebra that is a target of the registration, the three-dimensional target vertebra being extracted from a three-dimensional image including the spine of the subject acquired before the surgery on the subject.
